(19) United States
(12) Patent Application Publication
(10) Pub. No.: US 2023/0002713 A1
(43) Pub. Date: Jan. 5, 2023

(54) CELL CULTURE SYSTEM AND METHODS OF USING THE SAME

(71) Applicants: Jennifer Hui-Chun HO, Taipei (TW); Oscar Kuang-Sheng LEE, Taipei (TW)

(72) Inventors: Jennifer Hui-Chun HO, Taipei (TW); Oscar Kuang-Sheng LEE, Taipei (TW)

(21) Appl. No.: 17/756,150

(22) PCT Filed: Nov. 19, 2019
(86) PCT No.: PCT/CN2019/119485

§ 371 Date: May 18, 2022

(51) Int. Cl. C12M 3/06; C12M 1/22; C12M 1/32; C12M 1/00

(52) U.S. Cl. CPC C12M 23/16; C12M 23/10; C12M 23/12; C12M 23/34

(57) ABSTRACT

The present disclosure provides a cell culture automation system that provides enclosed culture conditions that may reduce the risk of contamination and automatically culture cells in large scale. Particularly, the cell culture system comprises (i) one or more removable microfluidic microwells and (ii) a culture device holding the microfluidic microwells, wherein each microfluidic microwell has one or multiple hollow units compartmentalized, and containing microfluidic channels with no bottoms throughout the microfluidic microwell, wherein the microfluidic channels contain one or more cell inlets.

### FIELD OF THE INVENTION

The present disclosure relates to a field of cell culture. Particularly, the present disclosure provides a microwell array for automation of cell culture.

### BACKGROUND OF THE INVENTION

Many clinical trials focusing on cell transplantation have reported promising results. Compared with conventional laboratory experiments, clinical trials normally require a large number of cells, which can create a novel challenge when culturing cells. Considering both safety and efficiency, present cell production technology remains immature. Clinical cell production involves many complicated and experience-based steps, including surgical tissue collection, sample treatments (dissection, dissociation, and dispersion), and cell seeding. Given the vast individual variation among patient cells, each of these steps for cell production is extremely difficult to perform stably using a standardized protocol. In most clinical cases, cell processing for regenerative cell therapy depends largely on the skill and experience of experts. Therefore, significant technological advances are needed for the industrialization of regenerative medicine.

U.S. Pat. No. 10,233,415B1 provides a microfluidics device for culturing cells, such as cardiomyocytes or cardiomyocyte progenitors; and methods of culturing cells using the device. US 20190185808 provides a cell culture system including: a culture unit including a culture tank for culturing a cell in a culture solution; an automatic cell culture device automatically controlling the culture of the cell in the culture unit; and a cell culture device for transport, controlling the culture of the cell in the culture unit at the time of transporting the culture unit. However, the cell culture devices cannot achieve automation of cell culture.

### SUMMARY OF THE INVENTION

In one aspect, the present disclosure provides a cell culture system, comprising (i) one or more removable microfluidic microwells and (ii) a culture device holding the microfluidic microwells, wherein each microfluidic microwell has one or multiple hollow units compartmentalized, and containing microfluidic channels with no bottoms throughout the microfluidic microwell, wherein the microfluidic channels contain one or more cell inlets.

In one embodiment, the cell culture system comprises (i) multiple removable microfluidic microwells and (ii) a culture device holding the microfluidic microwells, wherein each microfluidic microwell has one or multiple hollow units compartmentalized, and containing microfluidic channels with no bottoms throughout the microfluidic microwells, wherein the surface area of the microfluidic channels of the microfluidic microwells is gradually enlarged; wherein relative to the microfluidic channels of the multiple microfluidic microwells, which have the smallest surface area, the surface area of the microfluidic channels of the multiple microfluidic microwells exhibits a 2ⁿ, 3ⁿ or 4ⁿ increase in size, wherein n is an integer that is one less than the numbers of the multiple microfluidic microwells; and wherein the microfluidic channels contain one or more cell inlets.

In some embodiments, the culture device is a culture plate or culture flask.

In one embodiment, the hollow unit is in a pattern of circles or polygons having 3 to 8 angles. In some embodiments, the hollow unit is in a pattern of triangles, tetragons, pentagons, hexagons, octagons or enneagons. In a further embodiment, the hollow unit is in a pattern of hexagon.

In one embodiment, there are at least 3 removable microfluidic microwells. In one embodiment, there are at least 5 removable microfluidic microwells. In some embodiments, there are 3 to 15 removable microfluidic microwells.

In one embodiment, the multiple removable microfluidic microwells connect each other in the culture device.

In one embodiment, the microfluidic channel contains multiple cell inlets.

In one embodiment, the microfluidic channels of the hollow units are in fluidic communication with each other.

The present disclosure provides a method for culturing cells, comprising (i) loading cells to the cell inlet(s) on the microfluidic channels of removable microfluidic microwells of the cell culture system and (ii) culturing the cells under a condition suitable for the proliferation of the cells.

In one embodiment, the cells are anchorage-dependent cells. In some embodiments, the cells are stem cells, neural cells or fibroblasts.

In one embodiment, the cells are loaded to the cell inlet(s) on the microfluidic channels of the multiple microfluidic microwells, which have the smallest surface area.

In one embodiment, the cells are loaded in a density of 30% confluence.

In one embodiment, the cells are loaded to the cell inlet(s) on the microfluidic channels under a situation wherein the culture device is tilted. In some embodiments, the tilt angle is 120, 240 or 360 degree for triangles and hexagons or 90, 180, 270, 360 degree for tetragons and octagons.

In one embodiment, when cells reach to a required amount, antecedent microfluidic microwell is removed and subsequent microfluidic microwell is added into the culture device of the cell culture system of the present disclosure.

In one embodiment, the cell loading is performed under sterile conditions without any contact with an unsterile environment.

In one embodiment, the method can automatically culture cells in large scale.

In one embodiment, the method can be used for clinical scale cell expansion.

### BRIEF DESCRIPTION OF THE DRAWINGS

FIG. 1 shows a three-dimensional schematic representation of an embodiment of a microfluidic device.

FIG. 2 shows an overhead view of multiple removable microfluidic microwells.

FIG. 3 shows an assembly drawing of an embodiment of a microfluidic device.

FIG. 4 shows MSCs seeding with the same cell number (A) or with the same density (30%)-2 units (B) in the cell culture system of the invention. (A) With the same seeding cell number, total cell numbers on the cell culture system were higher than those in a conventional culture flask. (B) With the same initial seeding density (30% confluence), fewer seeding cell numbers were acquired for the culture flask, and the folds of cell numbers in the culture flask were higher than in a conventional flask after a 5-day culture.

FIG. 5 shows the phenotype of MSCs in a cell culture system of the present disclosure. Both cell culture system of the present disclosure and conventional (Ctrl) flask culturing maintain the MSC phenotyping with high positivity for CD90, CD105, CD73 and negativity for CD45, CD34, CD11b, CD19 and HLA-DR.

FIG. 6 shows tri-linage differentiation capacity of MSCs in a cell culture system of the present disclosure. MSCs cultured within the cell culture system of the present disclosure were subsequently stimulated to differentiate. Both the cell culture system of the present disclosure and tranditional (Ctrl) flask culture maintain the tri-differention ability of MSCs. Cells are stained positive with (A) alkaline phosphatase and alizarin red after 21 days of osteogenic induction. (B) oil red O after 21 days of adipogenic induction, and (C) alcian blue after 6 days of chondrogenic induction, respectively.

FIG. 7 (A) and (B) shows MSC enhancement of paracrine and autocrine in the cell culture system of the present disclosure. (A) Condition medium was collected when changing the microfluidic microwell. As for the conventional flask, condition medium was collected at the same time. The concentration of exosome in condition medium in the cell culture system and the conventional flask was measured ((A)-1 and (A)-2). (B) Expression of paracrine and autocrine related gene SDF-1, S1PR1, CXCR4 and VEGF was detected with QPCR.

### DETAILED DESCRIPTION OF THE INVENTION

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims of the present invention are approximately that can vary depending upon the desired properties sought to be obtained by the present invention.

As used herein and in the appended claims, the singular forms “a,” “an,” and “the” include plural referents unless the context clearly dictates otherwise.

As used herein, the term “in fluid communication with” or “fluidly coupled to/with” refers to two spatial regions being configured such that a liquid may flow between the two spatial regions.

Considering the safety and stability required for cell processing, automation of cell culture provides vast advantages for cell therapy. First, in the area of safety, human error, the risk of infectious contamination, or sample cross-contamination could be virtually eliminated. Second, the automation of cell processing promises decreased variability in each operation. Third, as automation hardware becomes more widespread, the operating cost will decrease to less than the cost of hiring skilled technicians and will result in more efficient production of primary cells.

Accordingly, the present disclosure provides a cell culture automation system that provides enclosed culture conditions that may reduce the risk of contamination and automatically culture cells in large scale. Particularly, the cell culture system comprises (i) one or more removable microfluidic microwells and (ii) a culture device holding the microfluidic microwells, wherein each microfluidic microwell has one or multiple hollow units compartmentalized, and containing microfluidic channels with no bottoms throughout the microfluidic microwell, wherein the microfluidic channels contain one or more cell inlets.

In case that multiple removable microfluidic microwells are used in the cell culture system, the system comprises (i) multiple removable microfluidic microwells and (ii) a culture device holding the microfluidic microwells, wherein each microfluidic microwell has one or multiple hollow units compartmentalized, and containing microfluidic channels with no bottoms throughout the microfluidic microwells, wherein the surface area of the microfluidic channels of the microfluidic microwells is gradually enlarged; wherein relative to the microfluidic channels of the multiple microfluidic microwells, which have the smallest surface area, the surface area of the microfluidic channels of the multiple microfluidic microwells exhibits a 2ⁿ, 3ⁿ or 4ⁿ increase in size, wherein n is an integer that is one less than the numbers of the multiple microfluidic microwells; and wherein the microfluidic channels contain one or more cell inlets.

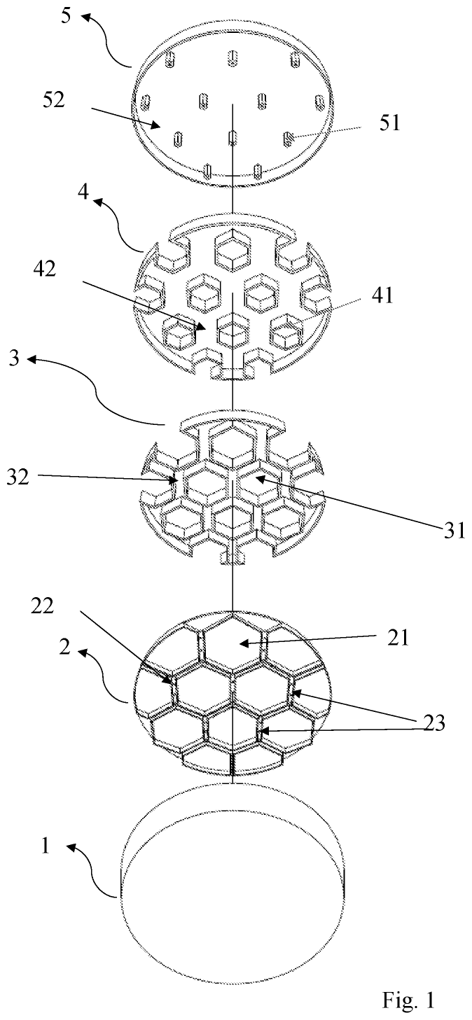

Referring now to FIG. 1, a three-dimensional schematic representation of an embodiment of a microfluidic device is shown. The depicted device includes a culture device 1 holding four microfluidic microwells 2, 3, 4 and 5. In each of the microfluidic microwell 2, 3, 4 and 5, it has multiple hexagon hollow units 21, 31, 41, 51 compartmentalized and contains microfluidic channels 22, 32, 42, 52 with no bottoms throughout the microfluidic microwell. The microfluidic channels 22 in the microfluidic microwell 2 have the smallest surface area; the surface area of the microfluidic channels 32, 42, 52 in the microfluidic microwell 3, 4 and 5 is 2 times, 4 times and 8 times of that of the microfluidic channels 22 in the microfluidic microwell 2, respectively. On the surface of each microfluidic channel, there are multiple cell inlets; an example of the cell inlets 23 is depicted in microfluidic microwell 2.

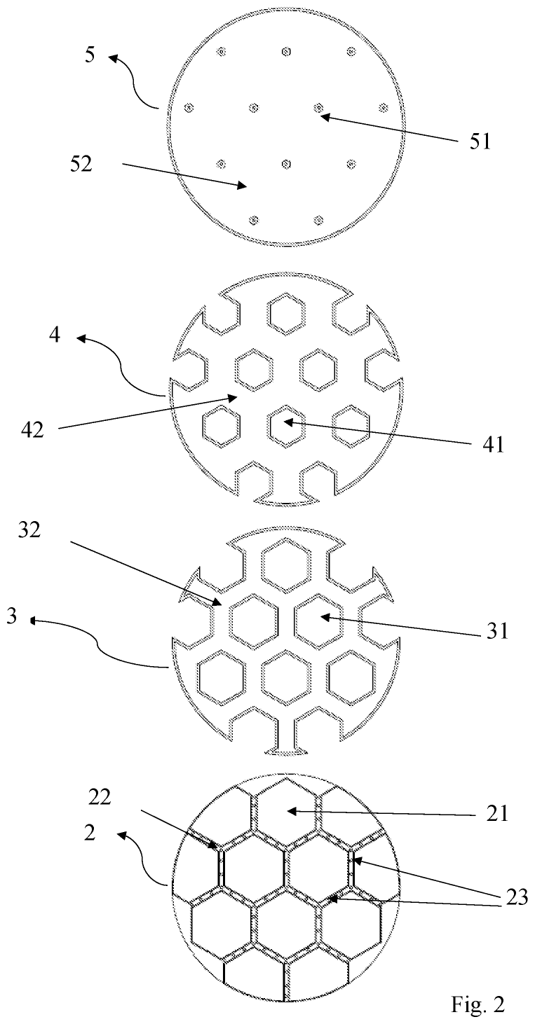

Referring now to FIG. 2, an overhead view of multiple removable microfluidic microwells 2, 3, 4 and 5 is shown. Each microfluidic microwell contains multiple hexagon hollow units 21, 31, 41, 51 and multiple microfluidic channels 22, 32, 42, 52. On the surface of each microfluidic channel, there are multiple cell inlets; an example of the cell inlets 23 is depicted in microfluidic microwell 2.

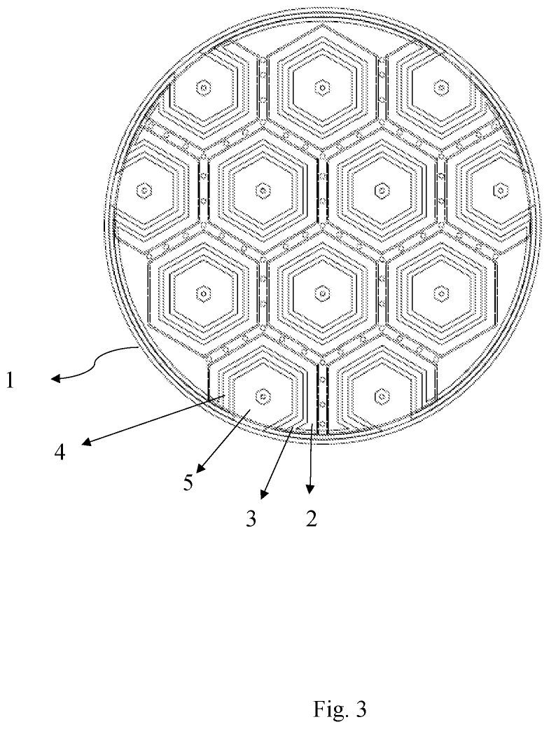

Referring now to FIG. 3, an assembly drawing of an embodiment of a microfluidic device is shown. The depicted device includes a culture device 1 holding four microfluidic microwells 2, 3, 4 and 5.

The culture device for holding the microfluidic microwells can be any suitable device. The culture device is used to hold one or more removable microfluidic microwells. The removable microfluidic microwell has one or multiple hollow units compartmentalized, and containing microfluidic channels with no bottoms throughout the microfluidic microwell. The microfluidic channels contain one or more cell loading inlets on the top thereof. The cells can be loaded to the microfluidic channels from the inlets. The microfluidic channels are in fluidic communication with each other so that the channels are full with medium for supporting growth and proliferation of the cells.

The hollow units can be on any suitable shape. For example, the unit can be patterned as circles, triangles, tetragons, pentagons, hexagons, octagons or enneagons.

In a preferred embodiment, multiple removable microfluidic microwells are used in the cell culture system. In such case, the surface area of the microfluidic channels of the microfluidic microwells is gradually enlarged and relative to the microfluidic channels of the multiple microfluidic microwells, which have the smallest surface area, the surface area of the microfluidic channels of the multiple microfluidic microwells exhibits a 2ⁿ, 3ⁿ or 4ⁿ increase in size. The number of n is one less than the numbers of the multiple microfluidic microwells. The multiple removable microfluidic microwells connect each other in the culture device. After the cells are loaded to the microfluidic channels of the removable microfluidic microwells, tilt or centrifuge the device to make cells even distribution in the microfluidic channels, and then grow and proliferate to required amount or density, the removable microfluidic microwells can be removed layer by layer so that the cells can be harvested. As such, the cells can be cultured automatically and replacing each individual manual step with a separate mechanized protocol is unnecessary.

The patterned microfluidic microwells can be produced using laser cutting technology to form various patterns (circle, triangle, square, hexagon, or octangon) with various sizes depending on user's need.

The following examples are offered to illustrate, but not limit the claimed invention.

### Example

Materials and Methods

Cell Culture System

The cell culture system includes multiple removable microfluidic microwells (such as four microwells) and a culture device holding the microfluidic microwells. The culture device holding the microfluidic microwells is a culture dish known used in cell culture field. The removable microfluidic microwells of the present disclosure were made with polydimethylsiloxane (PDMS). Using laser cutting technology, various patterns (circle, triangle, square, hexagon, or octangon) of various sizes were created in the bottom layer of the microfluidic microwell. A thin cover plate with a central hole for cell loading entrance was fixed on top of the pattern.

The cell-seeding process should take place under highly sterile conditions with no necessity for contact with an unsterile environment. The outer diameter is 150 mm, the height is 10 mm, and the cell culture medium volume is 1 mL. Centrifugation was performed to trap isolated individual and grouped primary cultured cells in the same dish. By utilizing this non-invasive system, long-term continuous monitoring, immediately following trapping, was made possible and cell growth and dynamics were successfully observed in the cell culture system. Media and liquid replacement does not require further centrifugation but instead utilizes capillary flow only. The scaffolds are inserted inside the chamber. The cell-seeding chamber is filled with culture medium, into which cells are injected through a cell culture inlet. The entire system can be fixed inside a regular humidified incubator. After seeding, the system, including the cell— polymer constructs, is transformed into a dynamic tissue cultivation system without exposing the constructs to an unsterile environment.

Cells

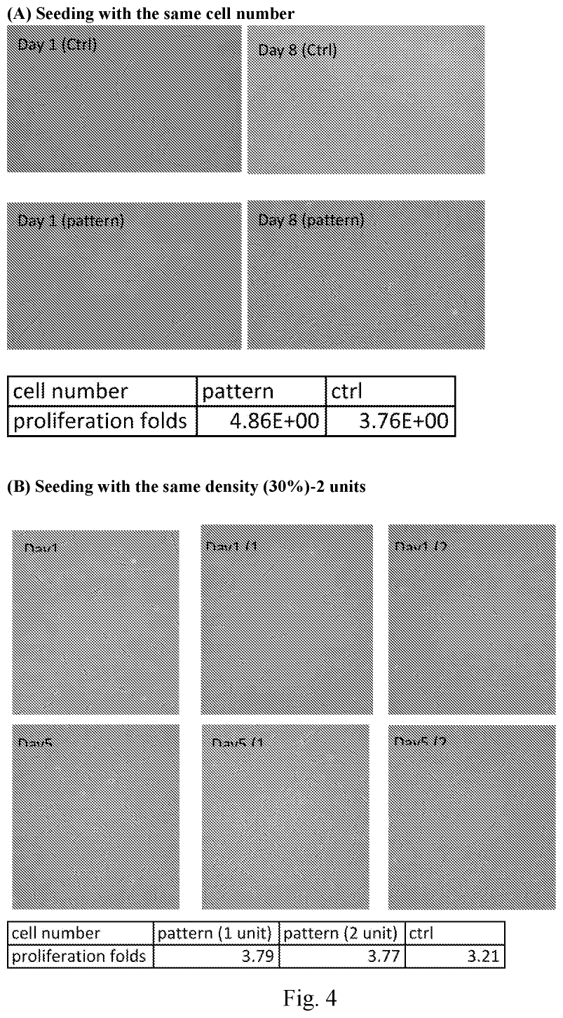

Mesenchymal stem cells (MSCs) were human orbital fat stem cells and maintained in their medium kits for growth (MesenPro). Cells possess the tri-linage differentiation ability and are positive for CD29, CD44, CD73, CD90, CD105, CD166 and negative for CD14, CD31, CD45 according to product instructions. As shown in FIG. 4, both the cell culture system of the present disclosure and conventional (Ctrl) culture flask maintain the MSC phenotyping with high postivity for CD90, CD105, CD73 and negativity for CD45, CD34, CD11b, CD19 and HLA-DR.

Cell Culture Chamber and Loading

Hexagonal-shaped microwells for cell culture. One thousand MSCs were seeded in a 15 cm culture dish within layer 1 pattern (L1). MSCs of the same number were seeded equally in a conventional 15-cm culture dish as control (Ctrl). As shown in FIG. 4, with the same seeding cell number, total cell numbers on the cell culture system exceeded those in conventional culture flask (A). With the same initial seeding density (30% confluence), lower seeding cell numbers were acquired for pattern culture flask, and the folds of cell numbers in the patterned culture flask exceeded those in the conventional flask after 5-day culture (B).

Flow Cytometry

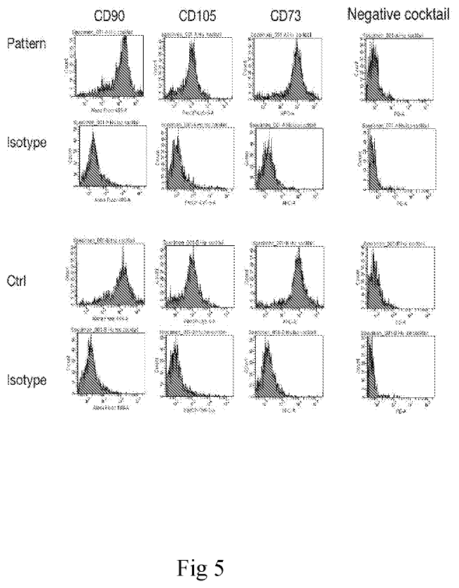

Flow cytometry assays were performed to characterize the proportion of MSCs in passage N cells. Cultured cells were harvested with 1 mM EDTA in PBS, centrifuged at 1,500 rpm for 5 min, and resuspended in 1 mL Memsen PRO. 1×10⁵ cells were transferred to a polystyrene round-bottom tube (BD Biosciences), centrifuged at 1,500 rpm for 3 min, and resuspended in 100 μL of FACS buffer containing monoclonal antibody (mAb). After incubation for 20 min at 4° C., the cells were washed with 1 mL FACS buffer and fixed in 300 μL of 1% paraformaldehyde in PBS. Fifty thousand cells per sample were acquired and analyzed using a FACSCanto II instrument (BD Biosciences) and Flow Jo. The expression of CD markers (CD44, CD73, CD90, CD105, CD11 b, CD19, CD34, CD45, and HLA-DR; BD Stemflow hMSC Analysis Kit; BD Biosciences, San Jose, Calif., USA). As shown in FIG. 5, both pattern and tranditional (Ctrl) flask culture maintain the MSC phenotyping with highly positive with CD90, CD105, CD73 and negative for CD14, CD34, CD11 and HLA-DR.

Confirmation of MSC Multipotentiality

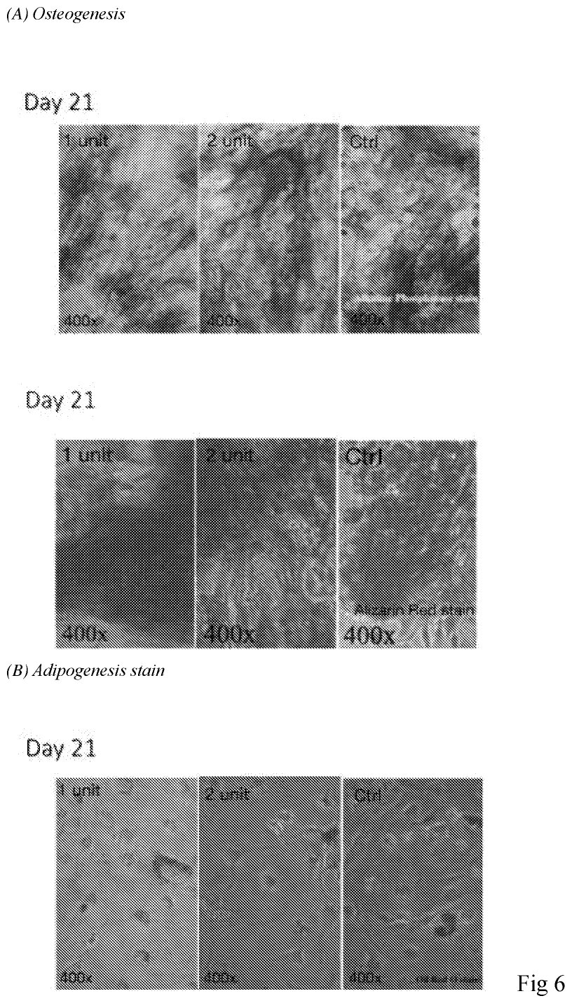

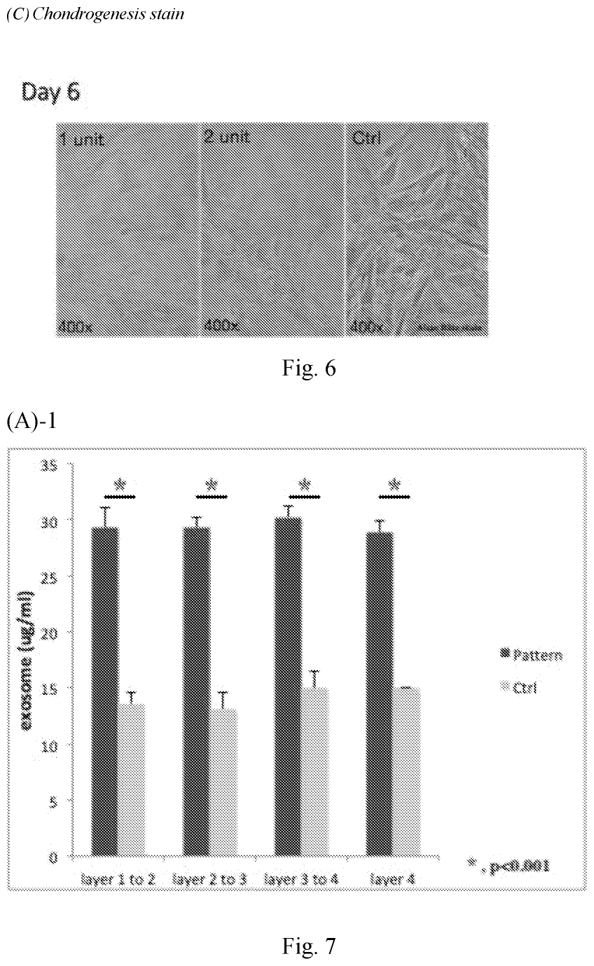

For adipogenesis, 1.9×10⁴ cells at passage 1 or 2 were plated in a 24 well-plate and cultured in 1 mL Memsen PRO. The medium was then switched to 1 mL complete STEMPRO adipogenesis differentiation medium (Invitrogen, Carlsbad, Calif., USA) once cells were 100% confluent. Cells were maintained in adipogenic medium for 3 weeks with medium changes twice weekly. The adipogenic cultures were fixed in 10% formalin (Sigma-Aldrich, St. Louis, Mo., USA) for 1 h at RT and stained with fresh Oil Red 0 solution (stock: 0.3% in isopropanol, mixed three parts stock to two parts water and filtered through a 0.2 m filter; Sigma-Aldrich) for 1 h at RT. Cells were then washed with water until the washes ran clear. Cells were visualized with a light microscope and photographed. To quantify adipogenic differentiation, the Oil Red 0 stain was eluted by adding 100% isopropanol (Sigma-Aldrich) for 10 min at RT. Absorbance at 490 nm was read in triplicates. For osteogenesis, 1×10⁴ cells were plated in a 24 well-plate and cultured in 1 mL Memsen PRO. The medium was replaced with 1 mL complete STEMPRO osteogenesis differentiation medium (Invitrogen) once cells were 50-70% confluent. Cells were maintained in osteogenic medium for 3 weeks with medium changes twice per week. The osteogenic cultures were fixed in 1 mL ice cold 70% ethanol (Sigma-Aldrich) for 1 h at 4° C. and stained with 4 mM Alizarin Red S in distilled water (pH adjusted to 4.2 with ammonium hydroxide; Sigma-Aldrich) for 10 min at RT. The excess dye was removed and washed four times with water. Cells were photographed with a light microscope. To quantify osteogenic differentiation, 400 mL of 10% (vol/vol) acetic acid was added to each well and incubated for 30 min with shaking. The cells were gently scraped with a cell scraper and transferred with 10% (vol/vol) acetic acid (Sigma-Aldrich) to a 1.5-mL microcentrifuge tube. The tube was sealed with parafilm, vortexed vigorously for 30 seconds, heated to 85° C. for 10 min and then transferred to ice for 5 min. After centrifugation at 20,000 g for 15 min, the supernatant was transferred to a new 1.5-mL microcentrifuge tube. The pH was adjusted to 4.1-4.5 with 10% (vol/vol) ammonium hydroxide (Sigma-Aldrich). Absorbance at 415 nm was read in triplicates. For chondrogenesis, 1.65×10⁵ cells were placed in a 15-mL conical tube and centrifuged at 1500 rpm for 5 min. The pellets were cultured in 0.5 mL of complete STEMPRO chondrogenesis differentiation medium (Invitrogen) for 1 week. Photographs of the pellets were taken with a ruler for size analysis. The pellets were fixed in 4% paraformaldehyde for up to 2 days and then placed in 1 mL 30% sucrose at 4° C. for 1 day. Cryosections (10 μm) were mounted onto slides and stained with Toluidine Blue O (Sigma-Aldrich). Photographs were taken with a light microscope. To quantify chondrogenic differentiation, pellets were fixed with 4% paraformaldehyde for 15 min, washed twice with 1×PBS, and stained with Toluidine Blue 0 for 15 min. The cells were washed again with 1×PBS to remove the unbound dye. The dye was extracted with 1% SDS and the absorbance at 595 nm was read in triplicates. As shown in FIG. 6, MSCs cultured within pattern were subsequently stimulated to differentiate. Both patterned and conventional (Ctrl) flask cultures maintained the tri-differention ability of MSCs. Cells were stained positive with (A) alkaline phosphatase and alizarin red after 21 days of osteogenic induction. (B) oil red 0 after 21 days of adipogenic induction, and (C) alcian blue after 6 days of chondrogenic induction, respectively.

Extraction of Exosomes from Culture Media of OFSC Cell Line

Exosomes were isolated from culture media using an isolation reagent (Exosome Isolation kit from culture media; Invitrogen, Carlsbad, Calif., USA) according to the manufacturer's protocol. The cells were seeded onto 10 cm dishes at a concentration of 1×10⁶ cells/dish with MesenPro containing 10% of exosome-free FBS (System Biosciences). After a 48-h incubation, conditioned media were harvested for exosome extraction. The culture medium was centrifuged at 2,000×g for 30 min to remove cells and debris. Then, the supernatants were passed through a 220 nm filter and transferred to new tubes, and the reagents were added. After incubation, the samples were centrifuged at 10,000×g for 1 h and the supernatants were discarded. The exosomes were pelleted at the bottom of the tubes and the pellets were resuspended in phosphate-buffered saline (PBS) for fluorescence imaging.

Quntitative PCR

The expression of autocrine and paracrine related genes were detected using QPCR. SDF-1 (F: 5′-GCCAAAAAGGACTTTCCGCT-3′(SEQ ID NO:1), R: 5′-GCCCGATCCCAGATCAATGT-3′(SEQ ID NO:2)).

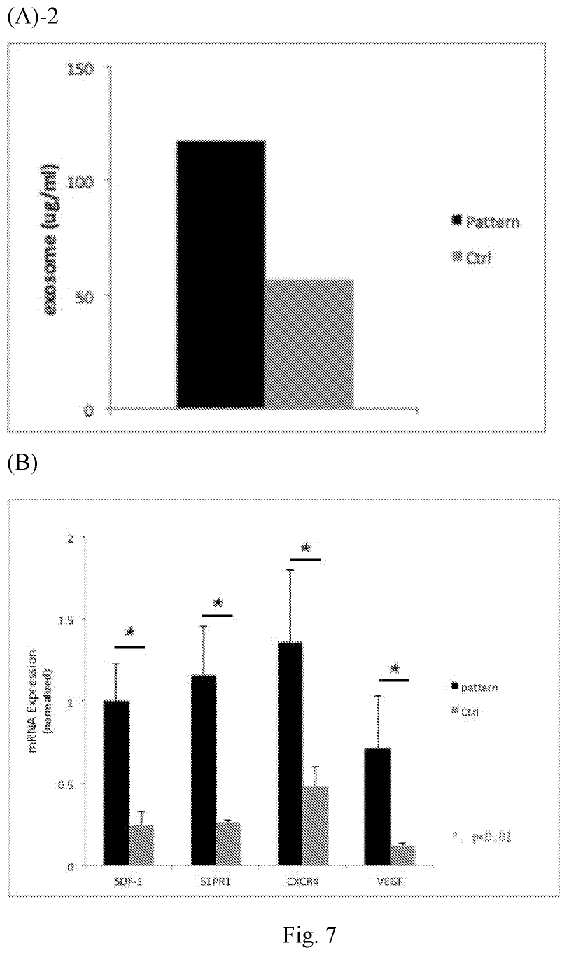

S1PR1(F:5′-TTTCCTGGACAGTGCGTCTC-3′ (SEQ ID NO:3), R: 5′-ACTGACTGCGTAGTGCTCTC-3′ (SEQ ID NO:4)). CXCR4 (F:5′-CGTCTCAGTGCCCTTTTGTTC-3′(SEQ ID NO:5), R:5′-TGAAGTAGTGGGCTAAGGGC-3′ (SEQ ID NO:6)). VEGF (F:5′-TACCGGGAAACTGACTTGGC-3′(SEQ ID NO:7), R: 5′-ACCACATGGCTCTGCTTCTC-3′(SEQ ID NO:8)). FIG. 7 shows that (A) condition medium was collected when changing the microfluidic microwell. For the conventional flask, condition medium was collected at the same timing. The concentration of exosome in condition medium in the cell culture system of the present disclosure was significantly increased than the concentration of exosome in condition medium in the conventional flask at each timepoint when changing microfluidic microwell, and the overall concentration of exosome in condition medium in the cell culture system was more than 2 fold of that in the conventional flask after changing microfluidic microwell for 4 times (7(A)-1 and 7A)-2). (B) Expression of paracrine and autocrine related gene SDF-1, S1PR1, CXCR4 and VEGF was detected with QPCR.

1. A cell culture system, comprising (i) one or more removable microfluidic microwells and (ii) a culture device holding the microfluidic microwells, wherein each microfluidic microwell has one or multiple hollow units compartmentalized, and containing microfluidic channels with no bottoms throughout the microfluidic microwell, wherein the microfluidic channels contain one or more cell inlets.

2. The cell culture system of claim 1, which comprises (i) multiple removable microfluidic microwells and (ii) a culture device holding the microfluidic microwells, wherein each microfluidic microwell has one or multiple hollow units compartmentalized, and containing microfluidic channels with no bottoms throughout the microfluidic microwells, wherein the surface area of the microfluidic channels of the microfluidic microwells is gradually enlarged; wherein relative to the microfluidic channels of the multiple microfluidic microwells, which have the smallest surface area, the surface area of the microfluidic channels of the multiple microfluidic microwells exhibits a 2ⁿ, 3ⁿ or 4ⁿ increase in size, wherein n is an integer that is one less than the numbers of the multiple microfluidic microwells; and wherein the microfluidic channels contain one or more cell inlets.

3. The cell culture system of claim 1, wherein the culture device is a culture plate or culture flask.

4. The cell culture system of claim 1, wherein the hollow unit is in a pattern of circles or polygons having 3 to 8 angles.

5. The cell culture system of claim 1, wherein the hollow unit is in a pattern of triangles, tetragons, pentagons, hexagons, octagons or enneagons.

6. (canceled)

7. The cell culture system of claim 1, wherein the system comprises at least 3 removable microfluidic microwells.

8. (canceled)

9. The cell culture system of claim 1, wherein the system comprises 3 to 15 removable microfluidic microwells.

10. The cell culture system of claim 1, wherein the multiple removable microfluidic microwells connect each other in the culture device.

11. The cell culture system of claim 1, wherein the microfluidic channel contains multiple cell inlets.

12. The cell culture system of claim 1, wherein the microfluidic channels of the hollow units are in fluidic communication with each other.

13. A method for culturing cells, comprising (1) loading cells to the cell inlet(s) on the microfluidic channels of removable microfluidic microwells of the cell culture system of claim 1 and (ii) culturing the cells under a condition suitable for the proliferation of the cells.

14. The method of claim 13, wherein the cells are anchorage-dependent cells, stem cells, neural cells or fibroblasts.

15. (canceled)

16. The method of claim 13, wherein the cells are loaded to the cell inlet(s) on the microfluidic channels of the multiple microfluidic microwells, which have the smallest surface area.

17. The method of claim 13, wherein the cells are loaded in a density of 30% confluence.

18. The method of claim 13, wherein the cells are loaded to the cell inlet(s) on the microfluidic channels under a situation wherein the culture device is tilted or centrifuged.

19. The method of claim 13, wherein the tilt angle is 120, 240 or 360 degree for triangles and hexagons or 90, 180, 270, 360 degree for tetragons and octagons.

20. The method of claim 13, wherein when cells reach higher than 50% confluence, antecedent microfluidic microwell is removed and subsequent microfluidic microwell is added into the culture device of the cell culture system of claim 1.

21. The method of claim 13, wherein the cell loading is performed under sterile conditions without any contact with an unsterile environment.

22. (canceled)

23. The method of claim 13, wherein the method can automatically culture cells in large scale.

24. The method of claim 13, wherein the method can be used for clinical scale cell expansion.
